(19) United States
(12) Patent Application Publication
(10) Pub. No.: US 2009/0030342 A1
(43) Pub. Date: Jan. 29, 2009

(54) APPARATUS AND METHOD FOR RELEASING A SAMPLE OF MATERIAL

(75) Inventors: Peggy-Jean P. Flanigan, Woodbury, MN (US); Bryan S. Behun, White Bear Lake, MN (US); Kevin M. Cummings, Little Canada, MN (US); Tushar A. Kshirsagar, Woodbury, MN (US); Tera M. Nordby, Woodbury, MN (US); Jeffrey D. Smith, Marine on St. Croix, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 11/829,637

(22) Filed: Jul. 27, 2007

(51) Int. Cl. A61B 10/02; C08J 5/24; C12M 3/00

(52) U.S. Cl. 600/572; 427/489; 435/286.7; 435/287.5

(57) ABSTRACT

A first aspect of the present invention provides for devices to facilitate the release of sample materials from sample acquisition devices. The device comprises an abrasion element comprising at least one constriction or projection. A second aspect of the present invention provides for methods in which to use the devices to facilitate the release of sample materials from a sample acquisition device. Optionally, the device may contain at least one reagent to facilitate the release and/or detection of a microorganism, or component thereof, in a sample. Preferably, the devices and methods may be used in conjunction with a liquid medium in which the sample may be further processed.

### BACKGROUND

Sample acquisition devices, such as swabs, are generally used in many industries for collecting a sample of material from a sample source. The sample acquisition device can include a hollow shaft including a distal end and a proximal end, and a sample-collecting region. The sample-collecting region is typically a complex surface, such as porous medium, attached to the distal end of the hollow shaft. Typically, the distal end and proximal end are open or include an opening. In the medical industry, the sample acquisition device may be used to gather a sample of biological material from a nose, ear, throat, or other sample source (e.g., a wound). Specifically, the shaft may be handled to position the porous medium in contact with the nose, ear, throat, or other sample source. In the food service industry, the shaft of the sample acquisition device may be handled to position the porous medium in contact with a food preparation surface, a food container, and the like. The samples collected by the sample acquisition device may then be analyzed for the presence of an organism (an “analyte”). The analysis may incorporate an assay.

Prior to the analysis of the sample, the sample is typically transferred from the sample acquisition device in order to place the sample in condition for analysis. In some methods, the sample acquisition device may be placed in contact with a slide or other laboratory apparatus in order to transfer at least some of the sample to the slide or other laboratory apparatus. In other methods, a fluid, such as a buffer solution, may be introduced into the proximal end of the hollow shaft of the sample acquisition device. The fluid then flows through the hollow shaft and exits through an opening at the distal end, contacting the sample as the liquid exits the hollow shaft and passes through the porous medium.

The efficiency of release of the sample from the porous medium into a liquid can affect the sensitivity of subsequent analyses. Thus, some methods use a mechanical vortex to wash the sample off the sample acquisition device. Although mechanical vortexing facilitates the release of analytes from a sample acquisition device, it requires specialized equipment and a source of electrical power. Occasionally, samples must be collected and analyzed in locations which lack the equipment, power source, and/or trained technicians.

For these reasons, there is a need for a device that can be used to collect and subsequently release a sample consistently, efficiently, and without the need for specialized powered equipment or highly-skilled technicians.

### SUMMARY

In one aspect, the present invention includes a sample collection and preparation device comprising a housing. The housing comprises a wall forming a lumen, a first end dimensioned to receive a sample-collecting region of a sample acquisition device, a second end, and an abrasion element. The abrasion element comprises a plurality of constrictions of the lumen, each constriction forming an opening. At least one constriction is positioned between the first end and the second end of the housing. The shortest distance across at least one opening is smaller than the largest width of the sample-collecting region.

In another aspect, the present invention includes a sample collection and preparation device comprising a housing and an abrasion element. The housing comprises a wall forming a lumen, a first end dimensioned to receive a sample-collecting region of a sample acquisition device, and a second end. The abrasion element comprises a helical projection and an opening bounded at least in part by the helical projection. The shortest distance across the opening is smaller than the largest width of the sample-collecting region.

In another aspect, the present invention includes a device for sample collection and preparation comprising a housing and an abrasion element. The housing comprises a wall forming a lumen, a first end dimensioned to receive a sample-collecting region of a sample acquisition device, a second end, and a longitudinal axis extending from the first end to the second end. The abrasion element comprises an opening bounded by at least one projection, wherein the at least one projection is oriented substantially parallel to the longitudinal axis. The shortest distance across the opening is smaller than the largest width of the sample-collecting region.

In another aspect, the present invention includes a device for sample collection and preparation comprising a housing and an abrasion element comprising at least one projection. The housing comprises a wall forming a lumen, a first end dimensioned to receive a sample-collecting region of a sample acquisition device, and a second end comprising the abrasion element.

In another aspect, the present invention includes an abrasion element to facilitate sample collection and preparation. The abrasion element comprises at least one projection, an opening dimensioned to receive a sample acquisition device comprising a sample-collecting region, and a tensioning element. The shortest distance across the opening is smaller than the largest width of the sample-collecting region.

In another aspect, the present invention includes an instrument for the detection or identification of a microorganism. The instrument comprises a sample preparation chamber comprising an abrasion element and a detection system to detect the presence of a microorganism or a component thereof.

In another aspect, the present invention includes a method of processing a sample. The method comprises providing a device comprising an abrasion element, providing a sample acquisition device containing a sample disposed thereon, and contacting the sample acquisition device with the abrasion element at least two times.

In another aspect, the present invention includes a method of making an abrasion element. The method comprises placing polymerizable silicone polymer in a housing, inserting an abrasion element template into the polymerizable silicone polymer, allowing the silicone polymer to substantially polymerize, and removing the abrasion element template from the silicone polymer.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

As used herein, “a,” “an,” “the,” “at least one,” and “one or more” are used interchangeably. Thus, for example, a housing that comprises “an” abrasion element can be interpreted to mean that the housing can include “one or more” abrasion elements that contact a sample acquisition device during use.

The term “and/or” means one or all of the listed elements or a combination of any two or more of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

The invention will be further explained with reference to the drawing figures listed below, where like structure is referenced by like numerals throughout the several views.

FIG. 1 shows a frontal exploded view of a device, with optional sample acquisition device and optional cap, according to one embodiment of the present invention;

FIG. 2 shows a longitudinal cross-sectional view of the device of FIG. 1 with a sample acquisition device inserted in the abrasion element according to one embodiment of the present invention;

FIG. 3 shows an upper perspective longitudinal cross-sectional view of a device comprising an alternative abrasion element in a housing with a sample acquisition device inserted therein according to one embodiment of the present invention;

FIG. 4A shows an upper perspective longitudinal cross-sectional view of a device comprising an alternative abrasion element in a housing with a sample acquisition device inserted therein according to one embodiment of the present invention;

FIG. 4B shows an upper perspective longitudinal cross-sectional view of a device comprising an alternative construction of the abrasion element of FIG. 4A according to one embodiment of the present invention;

FIG. 4C shows an upper perspective longitudinal cross-sectional view of a device comprising an alternative spatial arrangement of the projections comprising the abrasion element of FIG. 4A according to one embodiment of the present invention;

FIG. 4D shows an upper perspective longitudinal cross-sectional view of a device comprising an alternative abrasion element in a housing according to one embodiment of the present invention;

FIG. 5A shows an upper perspective longitudinal cross-sectional view of a device comprising a helical abrasion element in a housing according to one embodiment of the present invention;

FIG. 5B shows an upper perspective longitudinal cross-sectional view of a device comprising an alternative helical abrasion element in a housing according to one embodiment of the present invention;

FIG. 5C shows an upper perspective longitudinal cross sectional view of a device comprising an alternative helical abrasion element in a housing according to one embodiment of the present invention;

FIG. 6 shows an upper perspective longitudinal cross-sectional view of a device comprising an alternative abrasion element in a housing according to one embodiment of the present invention;

FIG. 7 shows an upper perspective, longitudinal cross-sectional view of an alternative abrasion element in a housing with a sample acquisition device inserted therein according to one embodiment of the present invention;

FIG. 8 shows an upper perspective, longitudinal cross-sectional view of an abrasion element according to the present invention with a sample acquisition device inserted therein according to one embodiment of the present invention;

FIG. 9 shows an upper perspective view of an alternative abrasion element according to one embodiment of the present invention;

FIG. 10A shows a top view of the device of FIG. 5A comprising a helical abrasion adjacent to a top view of a sample acquisition device according to one embodiment of the present invention;

FIG. 10B shows a top view of the device of FIG. 4B comprising a planar abrasion element adjacent to a top view of a sample acquisition device according to one embodiment of the present invention;

FIG. 10C shows a top view of the device of FIG. 4C comprising an abrasion element adjacent to a top view of a sample acquisition device according to one embodiment of the present invention; and

FIG. 10D shows a top view of the device of FIG. 6 comprising an abrasion element adjacent to a top view of a sample acquisition device according to one embodiment of the present invention;

FIG. 11A shows a longitudinal cross-sectional view of a housing with an upper perspective frontal view of an abrasion element comprising a projection according to one embodiment of the present invention;

FIG. 11B shows a longitudinal cross-sectional view of the housing of FIG. 11A with a side view of the projection according to one embodiment of the present invention;

FIG. 12A shows a longitudinal cross-sectional view of a housing with a frontal view of an abrasion element comprising a projection according to one embodiment of the present invention; and

FIG. 12B shows a longitudinal cross-sectional view of the housing of FIG. 12A with a side view of the projection according to one embodiment of the present invention.

### DETAILED DESCRIPTION

The present disclosure concerns substantially self-contained devices for preparing a biological sample for detecting an analyte, such as Staphylococcus aureus, wherein the devices comprise at least one abrasion element. The abrasion elements comprise one or more surfaces that can be brought into contact with a sample acquisition device, such as a swab, whereby the abrasion element facilitates the release of sample material from the sample acquisition device. In some embodiments, the abrasion element disrupts the adherence of sample material to the sample acquisition devices, such that the sample material is easily released from the sample acquisition device into a liquid suspending medium. In other embodiments, the abrasion element physically dislodges and removes the sample material from the sample acquisition device, without the use of a liquid suspending medium. The abrasion element can be formed or constructed in many ways, of which several exemplary embodiments are discussed in more detail below. Optional elements of the present invention include, but are not limited to, a sample acquisition device, a liquid sample-suspending medium, a reagent, and a cover or cap.

Sample acquisition devices, such as a swab, are routinely used to collect samples for chemical, biochemical, biological, or microbiological analyses of various materials or surfaces. The sample acquisition devices comprise a sample-collecting region, which is contacted with the material or surface to be analyzed. The sample-collecting region can comprise a molded material, such as plastic, a nonwoven fibrous material, such as rayon, nylon, cotton or polyester, or a foam material, such as polyurethane foam or a cellulose sponge. The most commonly used sample acquisition devices comprise a sample-collecting region, comprised of nonwoven fibrous materials, located at the tip of the device. The sample-collecting region accumulates sample material by, for example, adsorption, absorption, or physical entrapment.

At least one drawback to the use of sample acquisition devices comprising nonwoven fibers or foams is the inherent variability of such materials. For example, the fibers can exhibit various sizes, shapes, density, and spatial arrangement. Foams typically comprise hollow cells formed in a variety of shapes, sizes and spatial orientation. This inherent variability can affect the ease from which a sample is dislodged from the material. Furthermore, the release of the sample from the sample acquisition device can be affected by the skill and experience of the lab technician performing the procedure. This may introduce an additional element of variability to a test procedure. The present invention provides a number of devices and methods by which these elements of variability may be minimized, thereby providing consistent, efficient release of a sample from a sample acquisition device.

One aspect of the invention is to provide a device to improve the efficiency and/or consistency of the release of sample material from a sample acquisition device, without the requirement for the use of an electrically-powered machine, such as a vortex mixer. Another aspect of the invention is to provide a device to improve, without the requirement for the use of an electrically-powered machine, the homogeneity of a sample of biological material for analysis. Another aspect of the invention is to provide methods and devices to reduce the variability of the release of microorganisms from individual sample acquisition devices of similar constructions. Accordingly, such devices and/or methods can be used to prepare a biological sample for analysis by a number of techniques, which are discussed in further detail below.

The inventive device is a relatively simple device that allows a sample of material to be collected, prepared, and, optionally, tested for an analyte at or near the sample source. Rather than transferring the sample of material to an off-site laboratory for preparation and analysis, the present invention allows an operator to obtain a sample of material from a sample source, prepare the sample for analysis, and then test for the presence of an analyte at or near the sample source. This helps to decrease the waiting time necessary for a test result. The device or assembly may be sterilized by, for example, moist heat, dry heat, radiation, gaseous ethylene oxide, peroxides, and the like. Furthermore, the device may be disposable, which helps to provide a clean, if not sterile, device for each use.

Of course, the inventive device can also be used in a laboratory or other off-site setting. In these embodiments, the operator can obtain the sample and place the sample acquisition device into the housing. The operator can proceed immediately to prepare the sample for analysis and, subsequently, transport it to a laboratory or other setting for analysis. Alternatively, after collecting the sample, the device can be transferred to a laboratory or other setting where the sample can be prepared and analyzed.

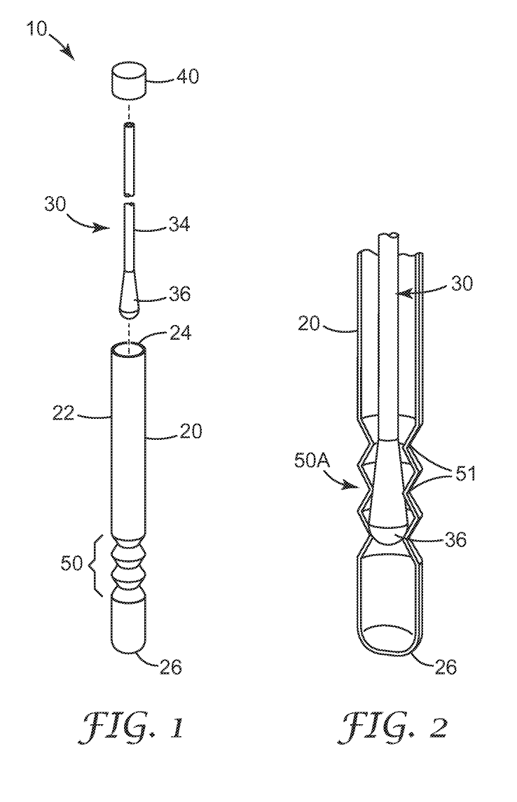

An exemplary device, according to the present invention, is shown in FIG. 1. This exploded view shows the sample processing device 10 is comprised of a housing 20, an optional sample acquisition device 30, and an optional cap 40. The housing comprises a wall 22 forming the outer perimeter of a lumen and two ends. The first end of the housing comprises an opening 24, which is dimensioned to receive the sample acquisition device 30, and a second end 26 opposite the first end. Preferably, the first end opening 24 is larger than the maximum outer dimension of the sample acquisition device, allowing for easy passage of the sample acquisition device 30 into the lumen formed by the walls 22 of the housing 20. In the embodiment shown in FIG. 1, the second end 26 is a closed end which may, optionally, hold a liquid sample-suspending medium. In other embodiments (discussed below), the second end may comprise an opening to facilitate the passage of sample material into a detection device or chamber, for example. The housing 20 further comprises abrasion element 50.

The optional sample acquisition device 30 comprises a shank 34 and a sample-collecting region 36. In this embodiment, the sample-collecting region 36 is located at the tip of the sample acquisition device 30. In other sample acquisition devices (not shown), the sample-collecting region may be located elsewhere. The sample-collecting region 36 can be formed from a number of materials and by a number of materials processes, as described above. Preferably, the sample-collecting region 36 is molded or adhered to the shank 34, such that the entire sample-collecting region 36 cannot be detached easily from the shank 34. The shank 34 can be substantially solid or, alternatively, relatively porous. In certain preferred embodiments, the shank 34 is hollow. The shank can be constructed from various materials, such as, for example wood, metal, or plastic.

The optional cap 40 is configured to fit over the first end opening 24 and may seal the housing 20 to protect the sample from the introduction of undesired contaminants and to minimize the loss of liquid or sample from the housing 20 during handling and/or transport. In certain embodiments, the cap 40 is attached to the shank 34, as indicated by a dashed line in FIG. 1. In some embodiments, the cap 40 is removably attached to the shank 34, such that the sample acquisition device 30 can be discarded after use and the cap 40 may be used to seal the housing for handling, transportation, or storage.

The abrasion element 50 may be of various sizes and/or shapes, examples of which are described below. One aspect of the abrasion element 50 is comprised of at least one surface that can contact the sample-collecting region 36 of a sample acquisition device. In certain embodiments, the abrasion element 50 may further comprise more than one surface that can contact the sample-collecting region 36 of a sample acquisition device. As discussed below, the contact surfaces may comprise projections, indentations, the walls 22 of the housing 20, or combinations thereof. In certain preferred embodiments, the shortest distance across an opening in the abrasion element 50 is smaller than the largest width of the sample-collecting region 36 of the sample acquisition device.

FIGS. 2-7 show longitudinal cross-sectional views of several exemplary illustrative embodiments of the present invention, each of which will be discussed in detail. The embodiments illustrated in FIGS. 1, 2, 6, and 7 are shown with a housing 20 comprising a closed end 26. Although only the embodiments illustrated in FIGS. 3-5 are shown with a housing 20 that is open at both ends, it should be noted that all abrasion elements in the present disclosure are compatible for use with a housing that comprises a first end opening 24 dimensioned to receive a sample acquisition device 30 and at least a small opening at the second end 26. Embodiments of a housing 20 that is open at both ends may be preferred in a sample-processing chamber of a system used for the detection or identification of a microorganism. An opening at the second end 26 may be large enough for the sample acquisition device 30 to pass therethrough. Alternatively, an opening at the second end 26 may be too small to allow the passage of the sample acquisition device 30, but large enough to allow a liquid medium to pass therethrough. An opening at the second end 26 may optionally comprise a removable watertight seal (not shown) to allow for temporary storage of liquid medium in the device until the seal is removed.

FIG. 2 shows a longitudinal cross-sectional view of the housing 20 of FIG. 1. In this embodiment, the abrasion element 50A comprises an accordion-like structure comprising constrictions 51 of the lumen, the constrictions 51 formed as indentations in the wall 22 of the housing 20. In this embodiment, the plurality of constrictions 51 forms a plurality of openings. Preferably, at least one opening formed by the constrictions 51 has a diameter that is smaller than the widest portion of the sample-collecting region 36 of the sample acquisition device 30. In this embodiment, the abrasion element 50A may provide circumferential contact with the sample-collecting region 36 of the sample acquisition device 30. Herein, circumferential contact means simultaneous contact between the abrasion element and the entire radial periphery of at least the widest portion the sample-collecting region 36. The constrictions 51 in FIG. 2 are shown as indentations that restrict the radial diameter of the entire circumference of the housing 20. Other embodiments (not shown) may have constrictions comprising indentations that restrict only part of the radial diameter of the housing 20.

FIG. 3 shows a longitudinal cross-sectional view of a housing 20 according to the present invention. In this embodiment, the abrasion element 50B comprises an opening bounded by a constriction comprising an annular ring 53, which is held in place by an anchor element 52. The anchor element 52 can be an integral part of the abrasion element 50B, as shown in FIG. 3, or it can be an integral part of the housing 20 (not shown). Alternatively, the anchor element 52 can be a separate part that is proportioned to be held by frictional force within the housing 20. Alternatively, the anchor element 52 may be bonded or adhesively secured to the housing 20. Similar to the constrictions 51 shown in FIG. 2, the annular ring 53 provides simultaneous contact with the entire radial periphery of at least the widest portion of the sample-collecting region, as it is moved through the annular opening 53.

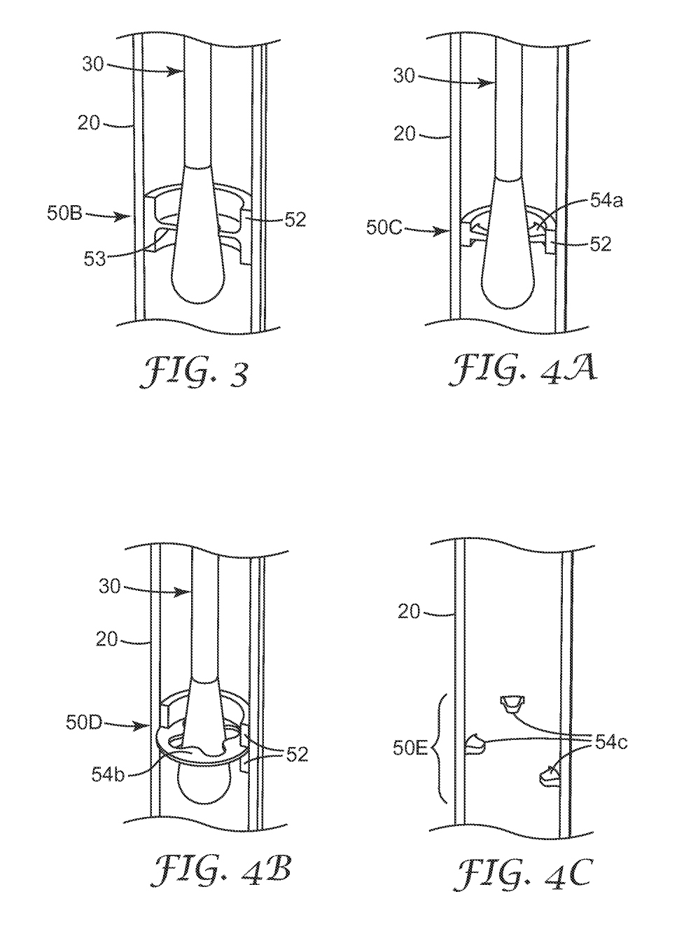

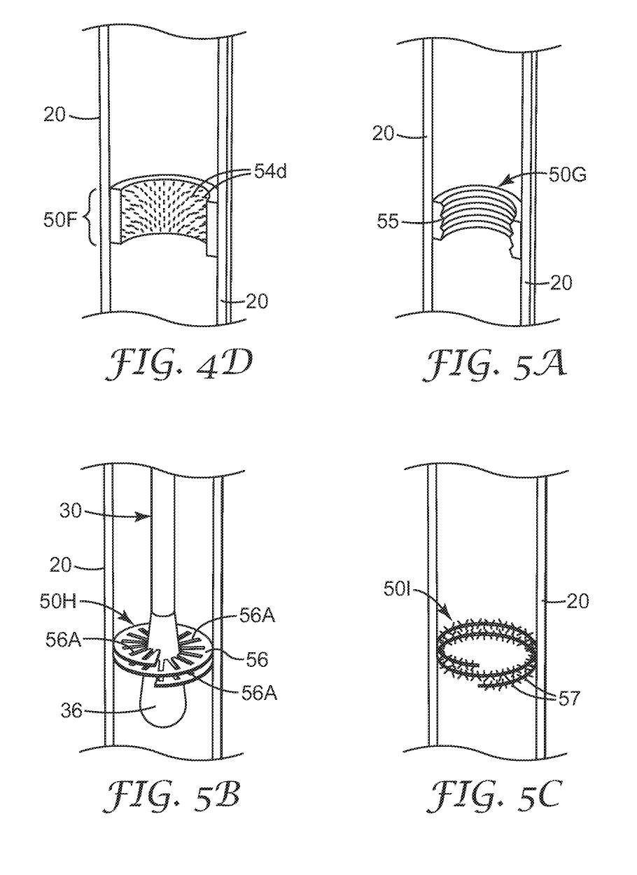

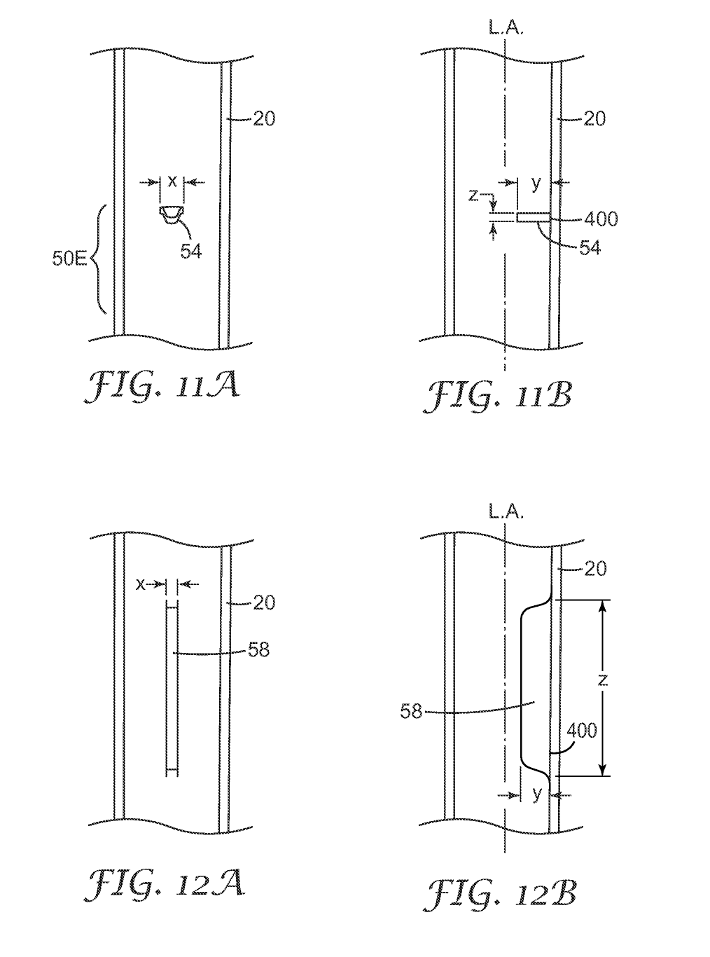

FIG. 4A shows a longitudinal cross-sectional view of a housing 20 comprising an alternative abrasion element 50C according to the present invention. In this embodiment, the abrasion element 50C comprises a plurality of projections 54a and an anchor element 52. In contrast to the abrasion elements 50A and 50B illustrated in FIGS. 2 and 3, respectively, the abrasion element 50C provides for discontinuous contact with the radial periphery of at least the widest portion of the sample-collecting region. That is, as the sample-collecting region is moved through the abrasion element 50C, the projections 54a do not provide for simultaneous contact with the entire radial periphery at least the widest portion of the sample-collecting region. The projections 54a can be configured in various shapes, sizes, and spatial arrangements. FIG. 11 illustrates certain spatial elements of the projections.

FIGS. 11A-B shows an upper perspective longitudinal cross-sectional view of a housing 20 comprising a single projection 54. The projection 54 is three-dimensional, having width (x), length (y), and depth (z), and is directly or indirectly associated with the wall of the housing 20 at the projection base 400. FIG. 11B also shows a dashed line marking an imaginary longitudinal axis (L.A.), extending from the first to the second ends of the housing 20. When either the width (x) or the length (y) is longer than the depth (z) of projection 54 or when the width (x) and length (y) are equal to the depth (z), the projection 54 is said to be oriented substantially perpendicular to the longitudinal axis (L.A.) of the housing 20.

FIG. 4B shows an alternative abrasion element 50D construction wherein the plurality of projections 54b are formed from an essentially planar material, which is held in place in the housing 20 by the anchor elements 52. Advantageously, the essentially planar material of abrasion element 50D can be manufactured to form the projections 54b by a die-cutting process.

Another embodiment, showing abrasion element 50E with an alternative configuration of projections, is shown in FIG. 4C, where the projections 54c are spaced apart at various positions around the inner perimeter and along the longitudinal axis of the housing 20. Optionally, the projections 54c may also be located at the bottom or second end, (not shown) of the housing 20. As shown in FIG. 4C, the housing 20 and abrasion element 50E are integrally formed as a single unit. Alternatively, the individual projections 54c could be formed into an abrasion element 50E comprising an anchor element 52, as shown in FIG. 4A (wherein the abrasion element 50C comprises a plurality of projections 54a).

Another embodiment, showing abrasion element 50F with an alternative construction of projections, is shown in FIG. 4D. In this construction, a piliated material, such as a textile comprising fibers or bristles 54d, is inserted into the housing 20. The piliated material may be bonded to the housing 20 to minimize movement of the abrasion element 50F during transportation, storage, or use. During use, the individual fibers or bristles 54d contact the sample acquisition device (not shown) to facilitate the release of sample material.

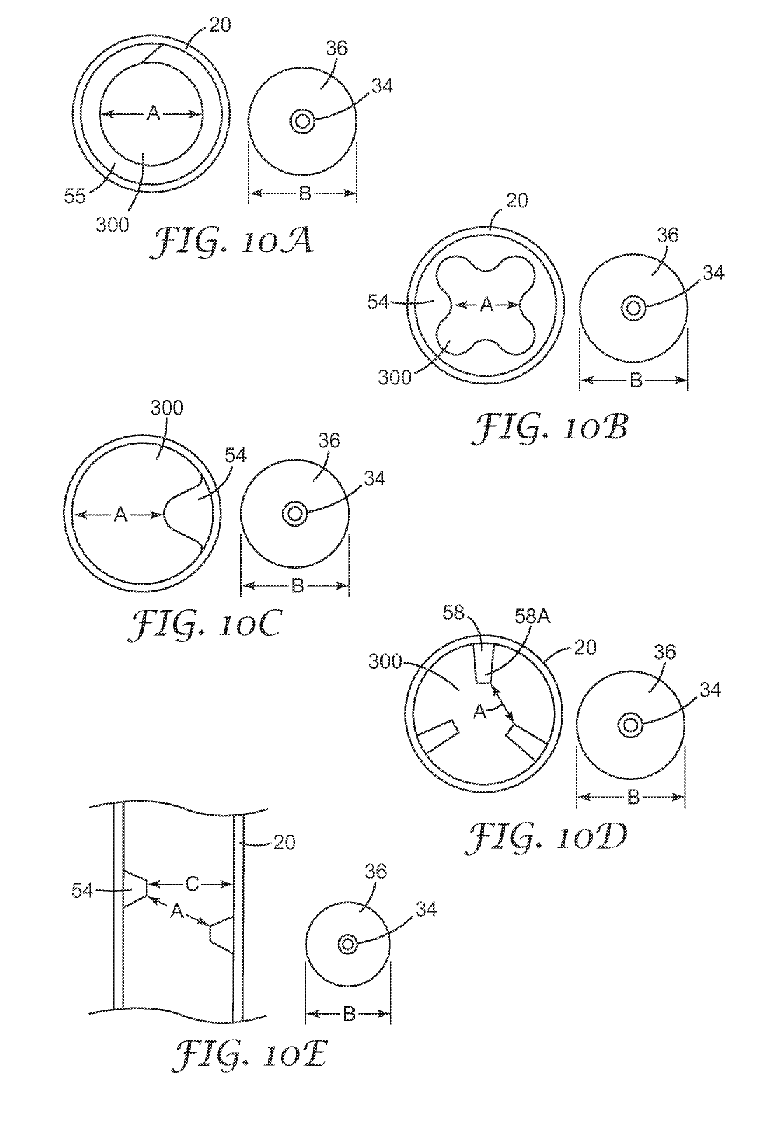

FIG. 5A shows a longitudinal cross-sectional view of a housing 20 comprising an alternative abrasion element 50G according to the present invention. In this embodiment, the abrasion element 50G comprises an opening bounded at least in part by a helical projection 55, as shown in FIG. 10A, wherein the opening 300 is bounded on all sides by the helical projection 55. The diameter of the opening 300 (shown as distance “A” in FIG. 10A) is preferably smaller than the largest width of the sample-collecting region 36 (shown as distance “B” in FIG. 10A) of the sample acquisition device 30. The abrasion element 50G may comprise a number of helical turns, or revolutions around the entire circumference of the inner surface of the housing. Preferably, the abrasion element 50G comprises at least 0.25 helical turns to 20 helical turns. More preferably, the abrasion element 50G comprises 2-12 helical turns. The pitch of the helical projection should be at least about 0.5 mm to about 5 mm. Certain embodiments comprise helical projections with a pitch of 1.59 to 3.2 mm. In certain embodiments, the abrasion element comprising a helical projection further comprises a threaded opening, such as a threaded nut.

FIG. 5B shows an alternative abrasion element 50H comprising a helical projection 56. In this embodiment, the helical projection 56 further comprises at least one notch 56A or, preferably, a plurality of notches 56A, as illustrated in FIG. 5B. The notches 56A interrupt the otherwise substantially linear edge of the helical projection 56 and may take the form of a variety of shapes or sizes. The notches 56A advantageously provide additional surface area to facilitate more contact with the sample acquisition device 20 and to promote better mixing when a liquid medium is present. Also, the notches 56A may allow for greater flexion of the helical projection 56 and thus provide for additional contact with the sample acquisition device 20 to remove the sample. Similar to the abrasion elements 50C-E, the abrasion element 50H may provide for discontinuous contact with the radial periphery of the widest portion of the sample-collecting region 36 of the sample acquisition device 30, as the sample-collecting region 36 is moved through the abrasion element 50H. The abrasion element 50H may be produced by die-cutting an appropriate material, such as, for example, a plastic film, and may be held in place in the housing 20 by friction, mechanical tension, bonding or by an adhesive, for example. A similar helical structure may be produced by arranging a plurality of individual projections in a helical arrangement (as shown in FIG. 4C). As used herein, “helical arrangement” means that at least three projections are distributed generally around the interior radial periphery of the housing and that they are also spaced apart along the longitudinal dimension of the housing 20. Although the plurality of projections may be evenly spaced apart, the helical arrangement does not necessarily require that the projections are distributed evenly along the radial periphery or in the longitudinal dimension. The material used to make the helical projections can be selected from a variety of materials, which embody a wide range of shapes, thicknesses, and flexibility, as discussed below.

FIG. 5C shows an upper perspective longitudinal cross-sectional view of a housing 20 with an alternative helical abrasion element 50I disposed therein. In this embodiment, the abrasion element 50I is coiled around the interior of the housing 20 and comprises bristles 57 which can contact a sample acquisition device (not shown) as it passes through the opening of the abrasion element 50I. Alternatively, the bristles 57 can be mounted in a non-helical support (not shown) that can be positioned in the housing 20 to contact the sample acquisition device.

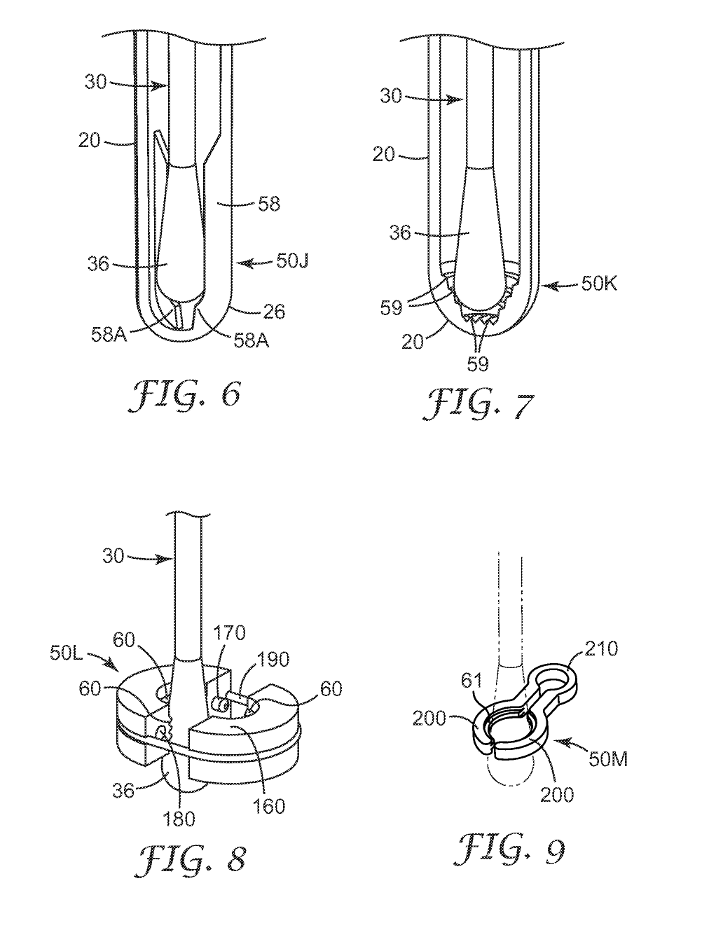

FIG. 6 shows a longitudinal cross-sectional view of the second end 26 of the device 10 with a sample acquisition device 30 inserted into the abrasion element 50J. The projections 58 can be configured in various shapes, sizes, and spatial arrangements. FIG. 12 A-B illustrates certain spatial elements of the projections.

FIGS. 12A-B shows an upper perspective view of longitudinal cross-sectional of a housing 20 comprising a single projection 58 according the embodiment of FIG. 6. The projection 58 is three-dimensional, having width (x), length (y), and depth (z), and is directly or indirectly associated with the wall of the housing 20 at the projection base 400. FIG. 12B also shows a dashed line marking an imaginary longitudinal axis (L.A.), extending from the first to the second ends of the housing 20. When both the width (x) and the length (y) are shorter than the depth (z) of projection 58, the projection 58 is said to be oriented substantially parallel to the longitudinal axis (L.A.) of the housing 20.

FIG. 6 shows the projections 58 positioned near the second end 26 of the housing 20. Alternatively, the projections 58 could be positioned near the first end opening 24 of the housing 20 or between both ends of the housing 20. Certain preferred embodiments comprise projections 58 comprising an extension 58A that functions to contact the tip of the sample-collecting region 36 of the sample acquisition device 30. Such extensions 58A may either be curved, as illustrated in FIG. 6, or more angular (not shown) and they function to increase the contact area with the sample acquisition device 30.

FIG. 7 shows a longitudinal cross-sectional view of the second end 26 of a housing 20 containing an alternative abrasion element 50K. In this embodiment, the abrasion element 50K comprises a plurality of projections 59 arranged along the inner wall of the second end 26 of the housing 20. The projections 59 provide a plurality of abrasion surfaces when the sample-collecting region 36 of the sample acquisition device 30 is rubbed against, or optionally rotated in contact with, the second end 26 of the housing 20. The number and shape of the projections 59 can be variable and the design should be selected to be compatible with the size and shape of the sample-collecting region 36 of the corresponding sample collection device 30. Angular projections 59, as shown in FIG. 7 provide for excellent abrasion of the sample-collecting region 36. However, rounded projections (not shown) may also provide sufficient abrasion or mixing, when a liquid is present, to release the sample. The abrasion element 50K comprises at least one projection and may comprise up to about 100, up to about 200, up to 300, or up to about 500 projections 57. The number of projections may be limited by the size of the projections and the size of the housing 20. As shown in FIG. 7, the abrasion element 50K can be integrally formed as a unit with the housing or, alternatively, an analogous structure can be inserted into the housing 20 to form an abrasion element 50K that would function similarly.

FIGS. 8 and 9 show embodiments of abrasion elements 50L and 50M, respectively, which can be incorporated into the housing 20 of a sample processing device 10. Alternatively, these or other abrasion elements could be incorporated into an integrated system for processing and analyzing a sample containing microorganisms.

FIG. 8 shows a perspective view of an abrasion element 50L, with a sample acquisition device 30 inserted therein, according to the present invention. The abrasion element 50L comprises abrasion mounts 160, alignment pins 170 with corresponding alignment holes 180, and a tensioning element 190. In this embodiment, the tensioning element 190 consists of an elastic material, such as an elastic band or a spring, positioned to apply enough force to urge the abrasion mounts 160 together. In certain embodiments, the tensioning element 190 allows for at least some movement of the abrasion mounts 160 as the sample acquisition device 30 passes therebetween and, thus, can function with sample acquisition devices 30 of various sizes and/or shapes. In some embodiments, the tensioning element 190 provides enough force to maintain a substantially fixed position of the abrasion mounts 160, relative to each other, as a sample acquisition device 30 passes therebetween.

FIG. 8 shows abrasion mounts 160 that comprise of a plurality of projections 60 which, when abrasion mounts 160 are brought together by the tensioning element 190, form an opening with a plurality of projections 60. Alternatively, at least one abrasion mount 160 may comprise at least one projection 60. Preferably, the width of the opening formed in abrasion element 50L by the at least one projection 60 is smaller than the maximum width of the sample-collecting region 36 of the sample acquisition device 30. In the illustrated embodiment of FIG. 8, the projections 60 provide for essentially continuous contact with the entire radial periphery of the widest portion of the sample-collecting region 36, as the sample-collecting region 36 is moved longitudinally through the abrasion element 50L. Alternatively, the projections 60 can have an equal or larger diameter than the sample-collecting region 36 of the sample acquisition device 30. In some embodiments, the abrasion element 50L is provided in a housing (not shown).

FIG. 9 shows a perspective view of an alternative abrasion element 50M according to one embodiment of the present invention. The illustrative abrasion element 50M comprises a unitary device comprising an abrasion mount 200 and a tensioning element 210. The abrasion element 50M further comprises at least one projection 61, positioned on the inner part of the abrasion mount 200, which contacts the sample-collecting region of a sample acquisition device (not shown) as it passes between the abrasion mounts 200. The projection 61 can be made of the same material as the abrasion mount 200 or, optionally, can be made of a more or less rigid material. The abrasion element 50M can be formed from relatively rigid or relatively flexible materials, such as metal or plastic materials. The tensioning element 210, which in this embodiment comprises a bend in the material from which the abrasion element is formed, maintains the abrasion mounts 200 in a substantially fixed position relative to each other. When an object, such as a sample acquisition device (not shown) is inserted between the abrasion mounts 200, the tensioning element 210 provides a resistive force to restrict the movement of the abrasion mounts 200 away from each other. Various amounts of tension can be applied by the tensioning element 210 to the abrasion mounts 200. In general, when less tension is applied to the abrasion mounts 200, the abrasion element 50M will accommodate a broader range of sizes of the sample-collecting regions used in sample acquisition devices. In some embodiments, the abrasion element 50M is provided in a housing (not shown).

### Construction of Abrasion Elements

Devices of the present invention comprise surfaces to contact a sample acquisition device to facilitate the release of sample materials. The surfaces may comprise the walls of a tube or housing and abrasion elements such as projections or protrusions, or combinations thereof. In some embodiments, the abrasion elements may be formed as part of the wall of the housing, as illustrated in FIGS. 4C, 6, and 7, using processes such as injection-molding, extrusion, or embossing, for example. In other embodiments, the abrasion elements may be formed as separate parts, as illustrated in FIGS. 3, 4A and 4B. The separate parts may be formed by, for example, extrusion, injection-molding, or die-cutting, for example. The separate parts optionally may be attached to a housing by, for example heat-bonding, welding, friction fit, snap fit, or adhesives. Alternatively, the parts may be constructed of appropriate materials and proportioned such that, once inserted into the housing, they are held in place by frictional resistance.

The abrasion element surfaces that will contact the sample acquisition device can either be generally rounded or angular. Abrasion elements with relatively sharp, angular edges are preferred for their ability to abrade or scrape the sample off the sample acquisition device and to wring liquids out of absorbent sample acquisition devices. Abrasion elements with rounded edges are useful to mix liquids into and wring liquids out of absorbent sample acquisition devices.

The abrasion elements may be formed from a number of materials. For example, the abrasion elements may be formed from plastics, polymers, glass, metal, cellulosic materials, ceramics, rubber, or combinations thereof. Suitable polymers include, but are not limited to, polyethylene, polycarbonate, polypropylene, polystyrene, polytetrafluoroethylene (PTFE), nylon, and polyesters. In some embodiments, the abrasion elements can be formed from relatively inflexible materials, such as hard plastics, glass, metal, reinforced cellulosic materials, or the like. In other embodiments, the abrasion elements can be formed from relatively flexible materials. The degree of flexibility will be influenced by the material, the density, and the thickness. The abrasion elements may also take a number of physical forms, such as projections, films, fibers, rods, wires, sheets, and the like. The surfaces of the abrasion elements may either be smooth, flat, and regular, or may be rough, textured, microstructured, undulating, and/or irregular. The materials used to form the abrasion elements should be amenable to the manufacturing processes and, optionally, sterilization processes. The materials used to construct the abrasion elements should not cause significant irreversible binding, adsorption, or entrapment of the microorganisms to be detected or analyzed in a sample.

One aspect of the invention includes a method of making an abrasion element. In one embodiment, a polymerizable material, such as silicone polymer dental impression material, is placed into a tube or housing. Prior to the completion of polymerization, an abrasion element template, such as a screw, is inserted into the silicone polymer material and the material is allowed to polymerize. The screw is removed from the polymer material after it has substantially polymerized. That is, the polymer has polymerized enough to retain the general shape, i.e., an opening and at least one projection, imparted by the abrasion element template. Optionally, the polymer material may be cut into sections, each section forming an abrasion element. The sections may be inserted into a tube or housing and used as an abrasion element as described below.

Without being bound by theory, applicants submit that sample release from a sample acquisition device is enhanced when there is physical comformance of the sample acquisition device to the abrasion elements, the abrasion elements to the sample acquisition device or conformance of both the sample acquisition device and the abrasion element to each other. As used herein, “physical conformance” means flexion of at least a part of the device to allow for more surface contact between the sample acquisition device and the abrasion element. The surface contact facilitates abrasion and removal of sample material from the sample acquisition device, and it facilitates mixing when a liquid is present. In some embodiments, at least a part of the abrasion element may be frangible, breaking away as the sample acquisition device passes by or through the abrasion element.

An advantage of the present invention is that the abrasion elements may be used in combination with one another and/or stacked to create a plurality of abrasion elements to enhance sample release. At least two, three, four, five, or more abrasion elements may be stacked to form a plurality of abrasion elements through which a sample acquisition device may be passed.

An advantage of the present invention is that the abrasion elements may comprise a coating. The coating may comprise reagents that interact with the sample, such as reagents for specimen storage or transport, reagents to adjust and/or maintain the pH of the sample, reagents to disrupt cells in the sample, reagents to release sample material from the sample acquisition device, reagents to detect a target analyte in the sample or a combination of any two or more reagents thereof. Reagents to adjust the pH of the sample may include buffering agents, such as sodium phosphate, potassium phosphate, TRIZMA, HEPES, sodium bicarbonate, buffered saline and the like. Reagents for specimen preservation or transport, such as Amies or Stuart's transport media may be coated in the device. Reagents to disrupt cells, such as an enzyme, an alkali, a surfactant, or a chaotroph may be coated on the device and may release target analytes such as a protein or nucleic acid from a cell to facilitate the detection of the target analyte. Enzymes for cell disruption include, for example, lysozyme, lysostaphin, trypsin, or protease K. In addition to facilitating the disruption of cell wall or cell membranes to release a target analyte, surfactants additionally may facilitate the release of sample material from the sample acquisition device. Nonlimiting examples of said surfactants include ionic surfactants, such as sodium dodecylsulfate or one or more of the following nonionic agents commonly available in surfactant tool kits: NINATE 411, Zonyl FSN 100, Aerosol OT 100%, GEROPON T-77, BIO-TERGE AS-40, STANDAPOL ES-1, Tetronic 1307, Surfynol 465, Surfynol 485, Surfynol 104PG-50, IGEPAL CA210, TRITON X-45, TRITON X-100, TRITON X305, SILWET L7600, RHODASURF ON-870, Cremophor EL, TWEEN 20, TWEEN 80, BRIJ 35, CHEMAL LA-9, PLURONIC L64, SURFACTANT 10G, SPAN 60, CREL; and combinations of any two or more of the foregoing.

Devices according to the present invention may comprise a housing and a liquid medium. In certain embodiments, the medium can be added to the housing of the device before a sample acquisition device is inserted therein. Alternatively, the medium can be added to the housing after insertion of the sample acquisition device. The volume of liquid medium may be adjusted so that at least one constriction, projection, or abrasion element may be in contact with the liquid medium during use, thereby facilitating the release of the sample material from the sample acquisition device. Alternatively, at least two, three, four, or more constrictions, projections, or abrasion elements may be in contact with the liquid medium. In certain embodiments, at least one, two, three, four, or more constrictions, projections, or abrasion elements are submerged in a liquid medium.

The liquid medium may comprise reagents that interact with the sample, described above, to adjust and/or maintain the pH of the sample, to preserve or transport the sample, to disrupt cells, to release sample material from the sample acquisition device, to detect a target analyte in the sample or combinations of reagents thereof. Nonlimiting example of reagents for target analyte detection include proteins, antibodies, enzymes enzyme substrates, oligonucleotides, particles and combinations of any two or more of the foregoing. Exemplary particles include polymeric, magnetic, paramagnetic, and silica particles, nanoparticles, and derivatives thereof.

### Microorganisms and Analytes

Microorganisms of particular interest for analytical purposes include prokaryotic and eukaryotic organisms, particularly Gram positive bacteria, Gram negative bacteria, fungi, protozoa, mycoplasma, yeast, viruses, and even lipid-enveloped viruses. Particularly relevant organisms include members of the family Enterobacteriaceae, or the family Micrococcaceae or the genera Staphylococcus spp., Streptococcus spp., Pseudomonas spp., Enterococcus spp., Salmonella spp., Legionella spp., Shigella spp. Yersinia spp., Enterobacter spp., Escherichia spp., Bacillus spp., Listeria spp., Vibrio spp., Corynebacteria spp. as well as herpes virus, Aspergillus spp., Fusarium spp., and Candida spp. Particularly virulent organisms include Staphylococcus aureus (including resistant strains such as Methicillin Resistant Staphylococcus aureus (MRSA)), S. epidermidis, Streptococcus pneumoniae, S. agalactiae, S. pyogenes, Enterococcus faecalis, Vancomycin Resistant Enterococcus (VRE), Vancomycin Resistant Staphylococcus aureus (VRSA), Vancomycin Intermediate-resistant Staphylococcus aureus (VISA), Bacillus anthracis, Pseudomonas aeruginosa, Escherichia coli, Aspergillus niger, A. fumigatus, A. clavatus, Fusarium solani, F oxysporum, F chlamydosporum, Listeria monocytogenes, Listeria ivanovii, Vibrio cholera, V parahemolyticus, Salmonella cholerasuis, S. typhi, S. typhimurium, Candida albicans, C. glabrata, C. krusei, Enterobacter sakazakii, Escherichia. coli O157 and multiple drug resistant Gram negative rods (MDR).

Gram positive and Gram negative bacteria are of particular interest for analytical purposes because there are a number of organisms within those groups that are known to be pathogenic to humans. Of even more interest are Gram positive bacteria, such as Staphylococcus aureus. Typically, these can be detected by detecting the presence of a cell-wall component characteristic of the bacteria, such as a cell-wall protein. Also, of particular interest are antibiotic resistant microbes including MRSA, VRSA, VISA, VRE, and MDR. Typically, these can be detected by additionally detecting the presence of an internal cell component, such as a membrane protein, transport protein, enzyme, etc., responsible for antibiotic resistance.

Analytes for detecting the organisms of interest include, for example, cell-wall proteins such as protein A and microbial surface components recognizing adhesive matrix molecules (MSCRAMMs) such as fibrinogen-binding proteins (e.g., Clumping Factor), fibronectin-binding proteins, collagen-binding proteins, heparin/heparin-related polysaccharides binding proteins, and the like. Protein A and Clumping Factor, such as fibrinogen-binding proteins and clumping factors A, B, and Efb, are also particularly useful in methods of detecting the presence of Staphylococcus aureus. Other external cell components of interest include capsular polysaccharides and cell-wall carbohydrates (e.g., teichoic acid and lipoteichoic acid).

### Samples and Sampling Techniques

Species of interest can be analyzed in a test sample that may be derived from any source, such as a physiological fluid, e.g., blood, saliva, ocular lens fluid, synovial fluid, cerebral spinal fluid, pus, sweat, exudate, urine, mucus, lactation milk, or the like. Further, the test sample may be derived from a body site, e.g., wound, skin, nares, scalp, nails, etc. The samples may consist substantially of solid, semi-solid, gelatinous, or liquid material, alone or in various combinations.

Samples of particular interest include mucus-containing samples, such as nasal samples (from, e.g., anterior nares, nasopharyngeal cavity, nasal cavities, anterior nasal vestibule, etc.), as well as samples from the outer ear, middle ear, mouth, rectum, vagina, or other similar tissue. Examples of specific mucosal tissues include buccal, gingival, nasal, ocular, tracheal, bronchial, gastrointestinal, rectal, urethral, ureteral, vaginal, cervical, and uterine mucosal membranes.

Besides physiological fluids, other test samples may include other liquids as well as solid(s) dissolved in a liquid medium. Samples of interest may include process streams, water, soil, plants or other vegetation, air, surfaces (e.g., contaminated surfaces), and the like. Samples can also include cultured cells.

The art describes various patient sampling techniques for the detection of microbes, such as S. aureus. Such sampling techniques are suitable for the methods of the present invention as well. For example, it is common to obtain a sample from wiping the nares of a patient. A particularly preferred sampling technique includes the subject's (e.g., patient's) anterior nares swabbed with a sterile swab or sampling device. For example, one swab is used to sample each subject, i.e., one swab for both nares. The sampling can be performed, for example, by inserting the swab dry or pre-moistened with an appropriate solution into the anterior tip of the subject's nares and rotating the swab for two complete revolutions along the nares' mucosal surface.

A wide variety of swabs or other sample acquisition devices are commercially available, for example, from Puritan Medical Products Co. LLC, Guilford, Me., under the trade designation PURE-WRAPS or from Copan Diagnostics, Inc. Corona, Calif., under the trade designation MICRORHEOLOGICS nylon flocked swab. A sample acquisition device such as that disclosed, for example, in U.S. Pat. No. 5,879,635 (Nason) can also be used if desired. Swabs can be of a variety of materials including cotton, rayon, calcium alginate, Dacron, polyester, nylon, polyurethane, and the like.

The sample acquisition device (e.g., swab) can then be cultured directly, analyzed directly, or extracted (e.g., by washing, elution by vortexing) with an appropriate solution. Such extraction (i.e., elution) solutions typically including water and can optionally include a buffer and at least one surfactant. An example of an elution buffer includes, for example, phosphate buffered saline (PBS), which can be used in combination, for example, with TWEEN 20 or PLURONIC L64. The test sample (e.g., liquid) may be subjected to treatment prior to further analysis. This includes concentration, precipitation, filtration, centrifugation, distillation, dialysis, dilution, inactivation of natural components, addition of reagents, chemical treatment, etc.

### Sample-Processing Methods

Sample processing devices of the present invention can be used in a number of methods for the detection of microorganisms in a sample. Sample-processing methods comprise providing an abrasion element according to the present invention, providing a sample acquisition device containing a sample disposed thereon, and contacting the sample acquisition device with the abrasion element. In some embodiments, the abrasion element is provided in a housing.

In a nonlimiting example of a sample-processing method, a sample acquisition device, such as a swab is used to collect a sample from, for example, the anterior nares of a patient. The swab is inserted into a device of the present invention, where the sample-collecting region of the swab is contacted with the abrasion element. Herein, “contacting” means moving at least a portion of the sample-collecting region of a sample acquisition device, or swab, into or through an opening formed by an abrasion element and/or moving the sample-collecting region against at least one projection of an abrasion element at least once. Where the device design allows it, it is preferable to move the swab sample-collecting region longitudinally through the opening formed by an abrasion element in a reciprocating motion for at least two or, more preferably, at least three or at least four, back-and-forth cycles to transfer by contact sample material from the sample acquisition device to the sample processing device. In certain embodiments, the swab sample-collecting region is rotated or vibrated against the abrasion element to facilitate the release of the sample material from the sample acquisition device.

In another embodiment, a sample acquisition device, such as a swab, is used to collect a sample. The swab is inserted into a device of the present invention and a sample-suspending liquid medium, such as phosphate buffered saline, is added to the device. In certain embodiments, the sample suspending-medium is passed through the lumen of the sample acquisition device into the device. The volume of the added sample-suspending medium may optionally, be large enough to contact or to at least partially submerge the abrasion element. After adding the sample-suspending liquid medium, the sample-collecting region of the swab is moved longitudinally through the abrasion element in a reciprocating motion for at least two, at least three, or at least four back-and-forth cycles. In certain embodiments, the swab sample-collecting region is rotated against the abrasion element to facilitate the release of the sample material. In alternative embodiments, the sample-suspending liquid medium is added to the device prior to inserting the sample acquisition device therein.

In some embodiments, the sample acquisition device is agitated, such as rotated or vibrated, while contacting an abrasion element. The agitation force may be applied to the sample acquisition device manually or by using a machine. In certain embodiments, the sample acquisition device, such as a swab, is attached to a device or machine that imparts rotational, vibrational, or reciprocating motion directly or indirectly to the swab as the swab is used with an abrasion element according to the present disclosure. Such devices and methods that use rotational, vibrational, or reciprocating motion to facilitate sample release are disclosed in U.S. patent application Ser. No. ______ (Attorney Docket No. 63035US002), filed on even date herewith, and entitled “SAMPLE RELEASE SYSTEM.”

### Sample Preparation Chambers

In the field of diagnostic microbiology, there are a number of instruments that are used to detect or identify target microorganisms. The instruments use a variety of technologies to detect the presence of whole organisms or sub-cellular component “analytes”, such as soluble proteins, lipoproteins, membrane-associated proteins, polypeptides oligopeptides, enzymes, cell wall-associated proteins, DNA, rRNA, mRNA, tRNA, oligonucleotides, adenosine triphosphate, polysaccharides, peptidoglycans, teichoic acids, lipotechoic acids.

The target analytes may be detected by immunological methods, through a binding reaction with specific antibodies, or genetic material specifying the target analyte may be detected by any known means for detecting DNA or RNA, such as genetic amplification (e.g., PCR, RT-PCR, LCR, and NASBA) or hybridization techniques. Non-limiting examples of such instruments used for target analyte detection include those described in the following patent publications: U.S. Pat. Nos. 6,889,468 and 7,056,473, and U.S. Patent Publication Numbers 2004/0137634A1 and 2005/0130177A1.

Devices of the present invention may be incorporated as a sample preparation chamber into an instrument used to detect microorganisms or components thereof. The sample preparation chamber may consist of any of the above-mentioned embodiments comprising an abrasion element. Preferably, the sample preparation chamber comprises a support structure for a chamber comprising a housing comprising wall, a lumen, a first end, a second end, and an abrasion element wherein the first end of the housing is dimensioned to receive a sample acquisition device comprising a sample-collecting region. Alternatively, the sample preparation chamber may comprise a support structure for an abrasion element. The support structure may be a simple shelf or holder of a suitable size and shape to hold the device or abrasion element. In certain preferred embodiments, the abrasion element or chamber is removable. In these embodiments, the abrasion elements or chambers may be disposable or, in certain embodiments, may be sterilized and re-used.

At least one function of the sample preparation chamber is to facilitate the release of the sample material from a sample acquisition device, as described above. Optionally, a liquid medium can be used in concert with the chamber. The liquid medium may contain reagents to further process the sample (e.g., release sample material, lyse cells, detect analytes), as described above.

Devices of the present invention may be used in an instrument with a detection system to detect the presence of a microorganism or a component of a microorganism in a sample. Suitable analytes for detection and exemplary detection instruments are described above.

### EXAMPLES

The present invention has now been described with reference to several specific embodiments foreseen by the inventor for which enabling descriptions are available. Insubstantial modifications of the invention, including modifications not presently foreseen, may nonetheless constitute equivalents thereto. Thus, the scope of the present invention should not be limited by the details and structures described herein, but rather solely by the following claims, and equivalents thereto.

The test tubes used to construct the devices described below were sterile, 12×75 mm test tubes, with cap, from VWR Scientific (West Chester, Pa.). Two lots of rayon-tipped swabs, of the design described in FIG. 2 of U.S. Patent Application Ser. No. 60/705,140 (Attorney Docket No. 61097US002), filed on Aug. 2, 2005, and entitled “APPARATUS AND METHOD FOR COLLECTING A SAMPLE OF MATERIAL” were obtained from Puritan Medical Products, Guilford, Me. The two different lots of swabs were manufactured on the same equipment using rayon fibers from the same lot. Staphylococcus aureus ATCC 6538 was obtained from the American Type Culture Collection (ATCC, Manassas, Va.). Phosphate Buffered Saline (PBS) consisted of 0.9% (w/v) NaCl in 10 mM sodium phosphate, pH, 7.4. PBS/L64 consisted of PBS containing 0.2% (w/v) Pluronics L-64 (BASF, Florham Park, N.J.).

### Preparative Example 1

### Swab Device with Single Blade

In the single-blade experiment, the collection test-tube was modified with a BD Bard-Parker™ “Rib-Back Carbon Steel #12 scalpel blade (BD Medical Systems, Franklin Lake, N.J.). The scalpel was positioned in the tube according to the following procedure. The plane the blade of the scalpel was oriented parallel to the longitudinal axis of the tube, with the blade edge pointed toward the bottom of the tube. The point of the scalpel was pierced through the wall of the test tube, at a height of approximately 1-3 cm above the bottom of the tube, until the point of the scalpel was located about 8 mm into the tube. The scalpel blade was glued into place with 5-Minute Epoxy (ITW Devcon, Danvers, Mass.). The exposed scalpel tip within the tube allowed the swab to be abraded when passed through the space between the scalpel blade and the opposite wall of the tube. In this construction, the swab was then rubbed up and down against the blade within the test-tube. The rubbing action resulted in moderate to severe physical destruction of the swab sample-collecting region, with release of some rayon fiber fragments from the swab sample-collecting region.

### Preparative Example 2

### Swab Device with Dual Blades

In the double-blade experiment, the collection test-tube was modified with two BD Bard-Parker™ “Rib-Back Carbon Steel #12 scalpel blades (BD Medical Systems, Franklin Lake, N.J.). The scalpels were positioned in the tube sequentially according to the following procedure. The plane of the blade of the scalpel was oriented parallel to the longitudinal axis of the tube, with the blade edge pointed toward the bottom of the tube. The scalpel was pierced through the wall of the test tube, at a height of approximately 1-3 cm above the bottom of the tube, until the point of the scalpel was located about 3 mm into the tube. The scalpel blade was glued into place with 5-Minute Epoxy (ITW Devcon, Danvers, Mass.). The blades were positioned across from each other (180 degrees) in the test tube. The exposed blade tips within the tube allowed the swab to be abraded when passed through the space between the scalpel blade tips. In this construction, the swab was rubbed up and down against the blade tips within the test-tube. The rubbing action resulted in moderate to severe physical destruction of the swab sample-collecting region, with release of rayon fiber fragments from the swab sample-collecting region.

### Preparative Example 3

### Swab Device with Threaded Nut

Nylon nuts (Nylon Lock Nut, 10-32, Part Number 0701032LN) were obtained from Micro Plastics, Inc. (Flippin, Ark.). The inner diameter (6.35 mm) of the nylon nuts was selected so that the sample-collecting regions of the swabs could move through the opening of the nut, with physical contact between the swab sample-collecting region and the inner (threaded) surface of the nut. The outer diameter (approximately 12.7 mm) of the hexagonal nuts was abraded, using a file and a scalpel, to shape them such that they fit into the plastic test-tubes. The screw pitch of the nylon nuts was 1.59 mm and the length of the longitudinal axis of the threaded opening was 6.35 mm (short nuts) or 12.7 mm (long nuts). The nuts were manually pressed into the test-tubes to a position approximately 1-3 cm above the base of the tube, such that the sample-collecting region of the swab could pass completely through the nut without hitting the bottom of the tube. The outer diameter of each abraded nut was closely matched to the inner diameter of the plastic tubes. Thus, the nylon nuts were held firmly in place by frictional resistance, without the need to use an adhesive or other elements to anchor the nuts. When using the nut to mechanically abrade the swab, the test tube was held tightly at the location of the nut, so that the nut would not slip during the procedure.

### Preparative Example 4

Swab device with threaded silicone-polymer nut.

Dental silicone impression material (Imprint III_198 VPS Heavy Body Impression Material) was obtained from 3M Company (St. Paul, Minn.). After test tubes were filled with the silicone, a screw was inserted. The “wide-thread” screws were pan head wood screws (51 mm long, 6.35 mm diameter, 1.59 mm screw pitch). The “narrow-thread” screws were pan head wood screws (51 mm long, 6.35 mm diameter, 3.2 mm screw pitch). After inserting the screws into the impression material, the polymer was allowed to cure at room temperature for approximately 1 hour. After the polymer was allowed to cure, the screw was removed and the tube was destroyed to obtain the silicone-molded part. Subsequently, a razor blade was used to cut the silicone polymer perpendicular to the threaded opening to create silicone nuts of various lengths. The longitudinal axis of the smaller silicone nuts was approximately 6 millimeters in length. The longitudinal axis of the larger silicone nuts was approximately 19 millimeters in length. Because the silicone polymer was cast in the same test tubes as the test tubes used for the bacterial extraction studies, the pieces were easily inserted into new tubes to create the swab device. The nut was pressed into a new test-tube and was held in place by the tight fit. When using the silicone polymer nuts to mechanically abrade the swab, the test tube was held tightly at the location of the nut, so that the nuts would not slip during the procedure.

### Example 1

### Microorganism Release from a Swab Using Vortex Action

The following method was devised to determine the percentage of organisms released from a swab. A colony of Staphylococcus aureus ATCC6538 was inoculated into a tube containing 10 mL of tryptic soy broth (TSB). The culture was incubated at 37 degrees C. for 18-24 hours. The culture was washed by centrifugation at approximately 12,000×g in PBS buffer, resuspended, and diluted in the same buffer to a concentration of approximately 1×10⁵ colony-forming units/milliliter (cfu/mL).

In all experiments, ten microliters of the diluted suspension was pipetted onto the sample-collecting region of each rayon swab (described above). The bacteria were removed from the swab sample-collecting region by using a 1 mL micropipetor to force 1 milliliter of PBS/L64 wash buffer through the lumen of the swab shank while the swab was held over a test tube. Hereinafter, this procedure of forcing buffer through the lumen of the swab to release loosely-attached microorganisms from the swab, will be called the “Push-Through” method. In some instances, shown below, the swabs were subsequently brought into contact with various abrasion elements to release bacteria from the swab sample-collecting region. Two 100 microliter aliquots of the buffer solution in the tube were each spread onto the surface of a petri dish containing blood agar (Tryptic Soy agar supplemented with 5% defibrinated sheep blood, Hardy Diagnostics, Santa Maria, Calif.) using a sterile plastic spreader and, subsequently, the plates were incubated overnight at 37° C. Bacterial colonies on each of the plates were counted and the counts were averaged for each swab sample. The number of organisms released from the swabs were compared to a control suspension that was added directly to a tube containing 1 milliliter of PBS/L64 buffer.

In the control suspension, ten microliters of the diluted bacterial suspension were pipetted directly into a tube containing one milliliter of PBS/L64. The control suspension was vortexed and the bacteria were enumerated as described for the swab samples.

### Example 2

### Comparison of Various Methods for Microorganism Release from a Swab

Suspensions of bacteria were prepared, applied to the swabs, and the swab sample-collecting regions were flushed with PBS/L64 as described in Example 1. The “Push-Through” experiments were performed, as described in Example 1, simply by forcing the wash buffer (PBS/L64) through the swab sample-collecting region to elute the bacteria from the fibers. The swab was not abraded with any device, nor was it pressed against the side of the tube to express any excess fluid trapped in the swab sample-collection region (bud). A control was run as described in Example 1. Two different lots of rayon swabs from Puritan Medical were tested. In the “Vortex” experiments, the swab was then immersed in the wash buffer after the buffer was forced through the lumen and into the tube. The tube was then vortexed for 30 seconds using an Analog Vortex Mixer (VWR Scientific) lab vortex, in order to release organisms that were remaining on the swab material. In the “Shaking” experiments, the swab sample-collecting regions were inoculated with bacteria and the buffer was forced through the lumen of the swab, as described in Example 1. Subsequently, the swab was immersed in the wash buffer and the tube was shaken by hand. In the shaking procedure, the tube was capped, and the tube was grasped firmly by the tube cap. At the beginning of each shake, the tube was held with its longitudinal axis about perpendicular to the ground. The bottom of the tube was then tipped at about a 45-degree angle toward the technician. The technician quickly rotated her wrist until the bottom of the tube was tipped at about a 45-degree angle away from the technician. This quick rotation of the wrist was repeated for the specified length of time and, when done properly, resulted in a mixing, or swirling, motion in the tube.

Five different methods for removal of the organisms from the swab were evaluated using the same initial suspension of bateria. The bacterial counts were compared to the control suspension, which was prepared and tested as described in Example 1. The results, which indicate that more bacteria were released from swab Lot 36505 regardless of the method used, are shown in Table 1.

TABLE 1
Efficiency of each method to release bacteria from swabs. The
effiency for each method is reported as a percentage of the
colony-forming units recovered from the swab wash solution. The
control bacterial suspension was the (100%) reference. The data
point for individual tests for each lot is an average of two bacterial
counts from one suspension recovered from a swab.

| | Push | | Shake | Shake | Shake |
|---|---|---|---|---|---|
| Swab Lot | Through | Vortex | (2 sec) | (16 sec) | (30 sec) |
| 36505 | 72 | 96 | 33 | 67 | 79 |
| 48105 | 35 | 92 | 14 | 42 | 53 |

### Example 3

### Release of Bacteria from Swabs Using Mechanical Abrasion

The following mechanical abrasion methods were used to determine how well they facilitated the release of bacteria from a swab sample-collecting region: blades to abrade/disintegrate the swab, a threaded-nut to rub the swab, and a tapered neck to squeeze the swab. The construction of the devices with single and double blades is described in Preparative Examples 1-2, respectively. The construction of devices with the Nylon Nut, which used the short nuts, is described in Preparative Example 3. The Puritan Medical swabs used for these experiments was swab lot 48105.

The “Lumen-Drip” method was similar to the “Push-Through” method described in Example 1 except that, after forcing the buffer through the lumen of the swab, the pipet tip was removed and any liquid remaining in the shank of the swab was allowed to flow out of the shank by gravity force. In typical experiments, this resulted in a release of from 0 to 150 microliters of additional buffer being released from the swab sample-collecting region.

The swabs were inoculated with 10 microliters of the bacterial suspension. Approximately one milliliter of PBS/L64 was used to elute the bacteria from each swab sample-collecting region by injecting the buffer through the swab shank. The buffer passed through the swab shank, out the holes in the shank and through the swab sample-collecting region into the test tube. The swab was then moved through the abrasion element in a reciprocating motion three times. With each of the reciprocating cycles, the entire sample-collecting region of the swab was passed completely through the abrasion element. On each downward passage through the abrasion element, the swab sample-collecting region was pushed into the buffer at the bottom of the tube. In order to keep the abrasion element from moving during the reciprocating motion of the swab, the tube was grasped at a position adjacent to the abrasion element during the procedure. It was observed that, in all cases, the abrasion procedure resulted in at least some mild destruction of the swab fiber, noted by the observation of loose fibers in the buffer. The percent of organism recovery for each of the respective abrasion elements is shown in Table 2.

TABLE 2
Efficiency of each method to release bacteria from swabs. The effiency for
each method is reported as a percentage of the cfu's recovered from the
swab wash solution. The control bacterial suspension was the (100%)
reference. The data point for individual tests for each lot is an average of
two bacterial counts from one suspension recovered from a swab.

| | | Vortex | Lumen | Shake | Double | Single | Nylon |
|---|---|---|---|---|---|---|---|
| | Control | (30 sec) | Drip | (30 sec) | Blade | Blade | Nut |
| Cfu | 1.2 × 10³ | 1.2 × 10³ | 1.0 × 10³ | 1.3 × 10³ | 1.2 × 10³ | 1.1 × 10³ | 1.2 × 10³ |
| % Release | 100 | 100 | 83 | 108 | 100 | 92 | 100 |

### Example 4

### Release of Bacteria from Swabs Using a Nylon Threaded-Nut

The bacterial suspension was prepared and deposited onto the swabs as described in Example 1. The control suspension was prepared and tested as described in Example 1. Swab devices with threaded nuts were prepared as described in Preparative Example 4, with the exception that the nylon nuts were pressed into a piece of clear polypropylene tubing, approximately 76 mm long. The bulk polypropylene tubing, which had a 12.7 mm outer diameter and a 9.5 mm inner diameter, was obtained from VWR Scientific and was cut to the appropriate length. The “bottom” end of the tubing was plugged with a sterile rubber stopper, to prevent leakage. The “No Swab (tube)” control was prepared and tested as described for the control suspension, except that the No Swab (tube) suspension was placed in the same tubing as the nylon nuts. The “Long Path” and “Short Path” abrasion elements were made from the “long” and “short” nylon nuts, respectively, as described in Preparative Example 3. After the PBS/L64 buffer was flushed through the swab sample-collecting region into the device, the swab sample-collecting region was passed through the threaded nut (back-and-forth) three times, for a total of six passages through the orifice of the nut. The percent recovery data are shown in Table 3. In contrast to abrasion methods shown in other examples, the threaded-nut type of abrasion demonstrated lower variability between the two different swab lots.

TABLE 3
Efficiency of bacteria release from swabs. The effiency for each
method is reported as a percentage of the cfu's recovered
from the swab wash solution. The control bacterial suspension
(No Swab) was the (100%) reference. The data point for
individual tests for each lot is an average of two bacterial counts
from one suspension recovered from a swab.

| | | | | Long | Short |
|---|---|---|---|---|---|
| Swab Lot | Control | No Swab (tube) | Vortex | Path | Path |
| 36505 | 100 | 95 | 102 | 76 | 87 |
| 48105 | 100 | 95 | 95 | 66 | 89 |

### Example 5

### Release of Bacteria from Clinical Swab Samples

The smaller threaded-nut devices from Preparative Example 3 were used in these experiments. Data from the two threaded devices represent replicate experiments using two devices of the same design. Swabs from two different lots were used to collect intranasal samples from three human subjects. Six samples were taken from each individual's nose using two different swab lots. The order in which the samples were taken from each subject's nose is shown in Table 4. After swabbing the anterior nares from a subject, the swab sample-collecting region was placed into the device and the swab sample-collecting region was flushed with PBS/L64, as described in Example 1. After subjecting the swab to the respective method for bacterial release, the liquid sample serially-diluted in PBS/L64 buffer and duplicate 100 microliter samples from each dilution were plated on CHROMagar Staphylococcus aureus medium (Hardy Diagnostics, Santa Maria, Calif.) and incubated at 37 degrees C. for the length of time specified by the manufacturer of the agar plates. After incubation, the Staphylococcus aureus colonies were counted according to the instructions provided by the manufacturer of the agar plates. For comparison, the bacteria were released from the swabs using vortex action, rather than by abrasion with threaded nuts.

TABLE 4
Order of samples taken from each subject's nose. All samples were
taken from both nostrils.

| Release Method | Swab Lot 36505 | Swab Lot 48105 |
|---|---|---|
| Threaded Device 1 | #1 | #2 |
| Vortex | #3 | #4 |
| Threaded Device 2 | #5 | #6 |

The average number of bacteria (cfu) released from each sample is shown in Table 5. Agar plates with 25 to 250 colonies were used to determine the number of bacteria a sample. The colony count was multiplied by the appropriate dilution factor to calculate the number of bacteria in the original nasal sample. The data indicate that methods employing the abrasion elements typically performed as well as or better than vortexing to release the clinical samples from the swabs. Although the number of samples was relatively small, the data indicate that the methods employing abrasion elements released the samples containing bacteria more consistently than the vortexing method.

TABLE 5
Bacteria release from clinical swabs. The number of bacteria, reported
in log cfu, is listed for each release method and swab lot The data
point for individual tests is an average of two bacterial counts from
one suspension recovered from each swab.

Swab Lot

| Subject | Method | 36505 | 48105 |
|---|---|---|---|
| #1 | Threaded Device 1 | 5.14 | 5.18 |
| | Vortex | 3.76 | 4.43 |
| | Threaded Device 2 | 4.47 | 3.72 |
| #2 | Threaded Device 1 | 3.30 | 3.24 |
| | Vortex | 2.35 | 2.35 |
| | Threaded Device 2 | 2.81 | 2.58 |
| #3 | Threaded Device 1 | 4.29 | 4.56 |
| | Vortex | 4.70 | 4.14 |
| | Threaded Device 2 | 4.20 | 3.90 |

### Example 6

### Release of Bacteria from Swabs Using a Silicone Polymer Threaded Nut

The performance of silicone nuts was compared to nylon nuts. Two silicone nut designs (wide-thread and narrow-thread, respectively) were prepared according to Preparative Example 5. The release of organisms from two swab lots was evaluated by pushing the swab bud completely through the nut, in a reciprocating motion, three times. A “No Swab” control was run to estimate the number of bacteria inoculated onto each of the swabs. In this control, a 10 microliter aliquot of the bacterial suspension was added directly to the PBS/L64 buffer and was vortexed for 30 seconds in a tube that did not contain an abrasion element or a swab. A “Silicone Plug” control was prepared by casting a plug of the VPS silicone polymer into the base of a test tube. This experimental control was performed like the No Swab control to assess nonspecific adsorption of bacteria to the silicone polymer. Organism release was measured as described in Example 1. The results are shown in Table 6 below. It was found that, on average, the silicone nut abrasion methods released approximately the same amount of bacteria as the vortexing method.

TABLE 6
Bacteria release from clinical swabs. The number of bacteria, reported
in log cfu, is listed for each release method and swab lot The data
point for individual tests is an average of two bacterial counts from
one suspension recovered from each swab.

| | Swab Lot | Swab Lot | |
|---|---|---|---|
| | 36505 | 48105 | Average |
| No Swab Control | NA | NA | 7.1 × 10² |
| Vortex 30 seconds | 8.1 × 10² | 7.4 × 10² | 7.8 × 10² |
| Silicone Plug Control | 8.9 × 10² | 7.3 × 10² | 8.2 × 10² |
| Silicone polymer nut, narrow-thread | 7.4 × 10² | 8.2 × 10² | 7.8 × 10² |
| Silicone polymer nut, wide-thread | 7.1 × 10² | 7.5 × 10² | 7.3 × 10² |
| Nylon nut | 7.5 × 10² | 6.0 × 10² | 6.8 × 10² |

The complete disclosures of all patents, patent applications, publications, and nucleic acid and protein database entries, including for example GenBank accession numbers, that are cited herein are hereby incorporated by reference as if individually incorporated. Various modifications and alterations of this invention will become apparent to those skilled in the art without departing from the scope and spirit of this invention, and it should be understood that this invention is not to be unduly limited to the illustrative embodiments set forth herein.

What is claimed is:

1. A device for sample collection and preparation, the device comprising:
a housing having
a wall forming a lumen;
a first end dimensioned to receive a sample-collecting region of a sample acquisition device;
a second end; and
an abrasion element comprising a plurality of constrictions of the lumen, each constriction forming an opening;
wherein at least one constriction is positioned between the first end and the second end of the housing; and
wherein the shortest distance across at least one opening is smaller than the largest width of the sample-collecting region.

2. The device according to either of claim 1, wherein at least one constriction provides circumferential contact with the sample-collecting region of the sample acquisition device.

3. A device for sample collection and preparation, the device comprising:
a housing comprising
a wall forming a lumen;
a first end dimensioned to receive a sample-collecting region of a sample acquisition device;
a second end; and
an abrasion element comprising a helical projection and an opening bounded at least in part by the helical projection; and
wherein the shortest distance across the opening is smaller than the largest width of the sample-collecting region.

4. The device according to claim 3, wherein the abrasion element comprises a plurality of projections in a helical arrangement.

5. A device for sample collection and preparation, the device comprising:
a housing comprising
a wall forming a lumen;
a first end dimensioned to receive a sample acquisition device comprising a sample-collecting region;
a second end; and
a longitudinal axis extending from the first end to the second end; and
an abrasion element comprising an opening bounded by at least one projection;
wherein the at least one projection is oriented substantially parallel to the longitudinal axis;
wherein the abrasion element is positioned between the first end and the second end of the housing; and
wherein the shortest distance across the opening of the abrasion element is smaller than the largest width of the sample-collecting region.

6. The device according to claim 5, wherein the abrasion element is positioned at the second end of the housing.

7. A device for sample collection and preparation, the device comprising:
a housing comprising
a wall forming a lumen;
a first end dimensioned to receive a sample acquisition device comprising a sample-collecting region;
a second end; and
an abrasion element comprising at least one projection;
wherein the second end of the housing comprises the abrasion element.

8. An abrasion element to facilitate sample collection and preparation, the element comprising:
at least one projection;
an opening dimensioned to receive a sample acquisition device comprising a sample-collecting region; and
a tensioning element;
wherein the shortest distance across the opening of the abrasion element is smaller than the largest width of the sample-collecting region.

9. The abrasion element according to claim 8 wherein the at least one projection is a helical projection.

10. The abrasion element according to any one of claims 5, 7, or 8 further comprising a plurality of projections.

11. The device according to any one of claims 1, 3, 5, or 7 further comprising a sample acquisition device.

12. The device according to any one of claims 1, 3, 5, or 7 further comprising a liquid medium contained within the housing.

13. The device according to any one of claims 3, 5, or 7, or the abrasion element of claim 8, wherein the abrasion element is die-cut.

14. An instrument for the detection or identification of a microorganism, the instrument comprising:
a sample preparation chamber comprising a device according to any one of claims 1, 3, 5 or 7, or the abrasion element of claim 13; and
a detection system to detect the presence of a microorganism or a component thereof.

15. A method of processing a sample comprising:
providing a device according to any one of claims 1, 5, 9, or 11;
providing a sample acquisition device containing a sample disposed thereon;
inserting the sample acquisition device into the housing; and
contacting the sample acquisition device with the abrasion element at least two times.

16. The method according to claim 15 further comprising the step of agitating the sample acquisition device.

17. The method according to claim 15 further comprising transferring a liquid medium into the housing.

18. The method according to claim 17, wherein the sample acquisition device, the abrasion element, and the liquid medium are in simultaneous contact.

19. The method according to claim 17, wherein the liquid medium comprises a reagent that interacts with the sample.

20. The method according to claim 15 further comprising detecting an analyte in the sample.

21. A method of processing a sample comprising:
providing an abrasion element according to claim 8;
providing a sample acquisition device containing a sample disposed thereon; and
contacting the sample acquisition device with the abrasion element at least two times.

22. The method according to claim 21 wherein the abrasion element is provided in a housing.

23. The method according to claim 22 further comprising transferring a liquid medium into the housing.

24. The method according to claim 23, wherein the sample acquisition device, the abrasion element, and the liquid medium are in simultaneous contact.

25. The method according to claim 23, wherein the liquid medium comprises a reagent that interacts with the sample.

26. The method according to any one of claims 21 or 23 further comprising detecting an analyte in the sample.

27. A method of making an abrasion element, the method comprising:
placing polymerizable silicone polymer in a housing;
inserting an abrasion element template into the polymerizable silicone polymer;
allowing the silicone polymer to substantially polymerize; and
removing the abrasion element template from the silicone polymer.
